(19) United States
(12) Patent Application Publication
(10) Pub. No.: US 2006/0052326 A1
(43) Pub. Date: Mar. 9, 2006

(54) Werner's syndrome helicase as a target for anti -HIV/AIDS therapy

(76) Inventor: Robert Harrod, Rockwall, TX (US)

Correspondence Address: BAKER BOTTS L.L.P., 2001 ROSS AVENUE, SUITE 600, DALLAS, TX, 75201-2980, US

(21) Appl. No.: 11/213,359

(22) Filed: Aug. 26, 2005

Related U.S. Application Data
(60) Provisional application No. 60/604,910, filed on Aug. 27, 2004.

(51) Int. Cl. A61K 48/00; A61K 38/16; A61K 31/551; A61K 31/522

(52) U.S. Cl. 514/44; 514/12; 514/220; 514/263.31

(57) ABSTRACT

Werner's Syndrome helicase (WRN) interacts in Tat/TAR-RNA chromatin-remodeling complexes assembled on the HIV-1 LTR to promote transcriptional activation and viral replication. WRN markedly increases basal HIV-1 transcription and synergizes with Tat to support LTR trans-activation. Inhibition of WRN functions, through expression of the trans-dominant-negative WRN_{K577M} mutant, potently represses HIV-1 LTR trans-activation and prevents intracellular synthesis of p24^{Gag} and inhibits virus production in HIV-infected H9_{HIV-1IIIB} lymphocytes. Therefore, the present disclosure provides a novel target for anti-HIV therapy. Methods and compositions of the disclosure may be used to perform high throughput screens for small molecule anti-HIV therapeutics. Such therapeutics may inhibit a stage in the HIV replicative cycle not affected by current anti-HIV drugs.

### RELATED APPLICATION

This application claims priority to U.S. Provisional Patent Application No. 60/604,910 filed on Aug. 27, 2004, the contents of which are hereby incorporated in their entirety by reference.

### FIELD OF THE INVENTION

The disclosure relates to the discovery that Werner's Syndrome helicase is an essential host cell factor in human immunodeficiency syndrome type 1 transcription and replication. The disclosure relates to systems, methods, and compositions for evaluating the interaction of Werner's Syndrome helicase with retroviral molecules, anti-HIV-1 therapeutic agents, and/or other molecules. The disclosure also relates to targeting WRN helicase in anti-HIV/AIDS therapeutic strategies.

### BACKGROUND

The Human Immunodeficiency Virus Type-1 (HIV-1) infects CD4⁺ lymphocytes and macrophages and causes immune-suppression associated with Acquired Immune-Deficiency Syndrome (AIDS). Approximately 41 million individuals are HIV-1-infected globally and nearly 1 million reside in the United States (Centers for Disease Control: HIV/AIDS Surveillance Report. Atlanta, Centers for Disease Control (1992); United Nations Development Programme: HIV/AIDS INDIA (2001); National Institutes of Health: Fiscal Year 2005 Plan for HIV-Related Research. Natural History and Epidemiology. Office of AIDS Research, United States Department of Health and Human Services, National Institutes of Health;). While modern combination anti-retroviral therapies have significantly reduced the number of HIV/AIDS-related deaths in the United States and Europe annually, impoverished or isolated areas without access to treatments remain devastated by the HIV/AIDS pandemic. Sub-Saharan Africa, including the Democratic Republic of Congo and Uganda, and southern Africa are among regions most affected by HIV/AIDS where nearly 25-35% of all adults and adolescents are HIV-positive (Centers for Disease Control: HIV/AIDS Surveillance Report. Atlanta, Centers for Disease Control (1992); United Nations Development Programme: HIV/AIDS INDIA (2001); National Institutes of Health: Fiscal Year 2005 Plan for HIV-Related Research. Natural History and Epidemiology. Office of AIDS Research, United States Department of Health and Human Services, National Institutes of Health;). Pediatric HIV-infections occur with high-incidence in sub-Saharan African countries and approximately 1,000 new cases are reported daily (Graham D I et al., Greenfield's Neuropathology, 7^{th} ed., pp 50-105). The Joint United Nations Program on HIV/AIDS has projected that densely-populated India and China will suffer major HIV-epidemics in the near future, as 5.8 million individuals are HIV-positive in Southeast Asia and 3.5 million HIV-infected people currently reside in India (United Nations Development Programme: HIV/AIDS INDIA (2001)).

### SUMMARY

During HIV-1 infection, the viral trans-activator, Tat, binds a uracil-containing RNA stem-loop (Tat-Activated Region-RNA, TAR-RNA) in HIV-1 transcripts to promote viral replication. However, numerous studies have demonstrated that HIV-1^{ΔTat} proviruses deleted for Tat sequences retain significant replication-competence, suggesting that cellular factors support basal HIV-1 replication.

The present disclosure provides, in some embodiments, factors that substitute for Tat functions. For example, Werner's syndrome (WRN) helicase may trans-activate an HIV-1 promoter. According to some embodiments, trans-activation may be dependent upon TAR-RNA and/or WRN-associated helicase activity and may synergize with Tat to promote activated HIV-1 transcription. In some specific example embodiments, immortalized human WRN^{-I-} fibroblasts, lacking a functional WRN helicase, are significantly impaired for basal and activated HIV-1 transcription, indicative that WRN may play an essential role in virus replication.

The present disclosure also provides, in some embodiments, agents that inhibit WRN functions including, without limitation, trans-activation of an HIV-1 promoter. For example, WRN functions may be inhibited by expression of a trans-dominant-negative mutant, WRN_{K577M}, which may potently repress both basal and Tat-activated HIV-1 transcription. WRN may co-localize with Tat in nuclei of HIV-infected tissues from donor AIDS patients. Moreover, a Tat mutant, Tat_{K28A/K50A}, defective for TAR-RNA-binding, may prevent WRN-mediated trans-activation from the HIV-1 LTR in some embodiments. According to these embodiments, WRN may be recruited to Tat/TAR-RNA-complexes through interactions that may promote “recycling” of the viral trans-activator. Inhibition of WRN may prevent intracellular synthesis of HIV-1 p24^{gag} and may inhibit viral replication in infected lymphocytes. These disclosures indicate that the WRN helicase may be an important and/or essential cofactor for HIV-1 transcription and/or viral replication in some embodiments. Inhibition of WRN functions or WRN-interactions with HIV components using agents of the present disclosure including, without limitation, small-molecule inhibitors, either alone or in combination with other anti-retrovirals, may be administered to ameliorate and/or prevent at least one symptom and/or complication of HIV/AIDS.

Some embodiments of the present disclosure may provide a novel target for anti-HIV/AIDS therapy, namely Werner's Syndrome (WRN) helicase. In addition to providing the WRN helicase itself as a target, embodiments of the present disclosure provide interactions between WRN helicase and retroviral molecules that may be targeted. For example, the interaction(s) between WRN helicase and Tat and/or TAR-RNA may be targeted. Without being limited to any particular model or mode of action, the present disclosure is based, in part, on the observation that cells that lack functional WRN helicase are significantly impaired for replication of HIV-1 or transcription of HIV-1 genes.

Embodiments of the disclosure also provide methods for identifying one or more molecules that block, impede, or otherwise interfere with WRN helicase-HIV-1 Tat interactions. Some embodiments provide, for example, a method of identifying a molecule that interferes with WRN helicase-HIV-1 Tat interaction. A method may include:

contacting a test molecule with a cell including:
a first nucleic acid having an HIV-1 long terminal repeat promoter operably linked to a reporter gene;
a second nucleic acid having a constitutive promoter operably linked to the Werner's Syndrome helicase gene; and
a third nucleic acid having a constitutive promoter operably linked to an HIV-1 Tat gene, wherein the constitutive promoter of the third nucleic acid may be the same or different from the constitutive promoter of the second nucleic acid;

cultivating the cell under conditions that permit expression of the reporter gene and the helicase gene in the absence of the test molecule;

detecting the reporter gene expression in the presence of the test molecule; and

comparing the reporter gene expression in the presence of the test molecule with reporter gene expression in the absence of the test molecule,

wherein reduced reporter gene expression in the presence of the test molecule relative to reporter gene expression in the absence of the test molecule indicates inhibition of WRN helicase-HIV-1 Tat interaction. In some embodiments of the disclosure, the reporter gene product is directly or conditionally lethal to the cell and the presence of an anti-HIV candidate drug is indicated by survival of the cell. In some embodiments, the identifying methods may be practiced as high-throughput screens.

Embodiments of the present disclosure also provide methods of blocking, impeding, or otherwise interfering with the interaction(s) between WRN helicase and HIV-1 Tat and/or TAR-RNA. For example, one embodiment provides a method of interfering with WRN helicase-Tat interaction in a cell. The method includes contacting the cell with a test molecule (e.g. nucleic acid, polypeptide, or organic molecule), wherein the test molecule inhibits WRN helicase-Tat interaction. In some embodiments of the disclosure, the organic molecule may be a small molecule drug. An example of a molecule that interferes with WRN helicase-Tat interaction is the trans-dominant-negative mutant, WRN_{K577M}.

### BRIEF DESCRIPTION OF THE DRAWINGS

The patent application file contains at least one drawing executed in color. Copies of this patent or patent application publication with color drawing(s) will be provided by the United States Patent and Trademark Office upon request and payment of the necessary fee.

Some embodiments of the present disclosure may be further described by referring, in part, to the instant description and accompanying drawings.

FIG. 1 shows HIV-1 LTR transcription complex interactions. The trans-activator Tat binds the TAR-RNA stem-loop element within viral transcripts and recruits the co-activators/acetyltransferases, p300/CBP and P/CAF; hGCN5, to the HIV-1 promoter. Acetylation of Tat on lysine K28 by P/CAF facilitates recruitment of the PTEF-b (cyclin T1-cdk9) complex that hyper-phosphorylates RNA Pol II on hepta-serines within its COOH-terminus. The locations of the two NF-κB (p65/p50)-responsive enhancer elements and three SP-1-responsive enhancer elements are shown; and the relative positions of the three nucleosomes (NUC 0, NUC 1, NUC 2) spanning the HIV-1 LTR are indicated below.

FIG. 2 shows that WRN and BLM helicases share significant amino acid sequence homologies with RNA-dependent helicases from different organisms. The exonuclease domain of WRN and conserved seven helicase repeats in WRN and BLM are shown. The substitution mutation, K577M, that inactivates WRN helicase activity, resulting in production of a trans-dominant-negative protein is indicated (Bai Y et al., Hum. Genet. 113:337-347). NLS, nuclear localization sequence.

FIG. 3 shows that the WRN protein transcriptionally activates the HIV-1 LTR dependent upon WRN-associated helicase activity.

FIG. 4 shows WRN-mediated trans-activation from the HIV-1 LTR is dependent upon TAR-RNA.

FIG. 5 shows that the WRN helicase trans-activates the HIV-1 LTR in Jurkat E6.1 lymphocytes. Error bars representative of standard deviations are shown.

FIG. 6 shows that immortalized human WRN^{-I-} fibroblasts are significantly impaired for basal and Tat-activated HIV-1 transcription.

FIG. 7 shows that inhibition of WRN functions significantly prevents intracellular synthesis of p24^{Gag} and inhibits HIV replication in infected H9 lymphocytes.

FIG. 8 shows that the WRN helicase is present in Tat-containing chromatin- remodeling complexes recruited to the HIV-1 LTR.

FIG. 9 shows that Tat and WRN co-localize in HIV-1-infected CNS-tissues from donor Neuro-AIDS patients.

FIG. 10 shows detection of purified recombinant GST, GST-HIV-1 Tat, and GST-HIV-1 Tat_{K28A/K50A} proteins by immunoblotting using an anti-GST goat polyclonal antibody.

### DETAILED DESCRIPTION

In addition to causing AIDS, HIV-1-infections of the central nervous system (CNS) result in primary encephalopathies and neuropsychological symptoms associated with AIDS-dementia complex. The neurological manifestations of HIV-infections typically coincide with late-stage AIDS and vary from mild cognitive disorders to a paralytic or complete vegetative state. Nearly one-third of all HIV-infected patients develop Neuro-AIDS at some point during the clinical course of disease progression (Simpson D et al., Med. Clin. North Am. 80:1363-1394; Janssen R S, AIDS and the nervous system. 2^{nd} ed., pp 13-37; Levy R et al., J. Neurosurg. 62:475-495; Burns D et al., Arch. Pathol. Lab. Med. 115:1112-1124; Brew B J et al, Neurologic Clinics 17:861-881; Sacktor N et al., Neurology 56:257-260; Dore G J et al., AIDS 13:1249-1253), although many individuals report neurological complications as their primary symptom prior to immune-suppression (Skiest D J, Clin. Infect. Dis. 34:103-115). The consequences of Neuro-AIDS are most apparent during pediatric HIV-infections, which may lead to mental retardation or severe learning deficiencies frequently accompanied by neurophysical impairments, such as blindness and/or hearing-loss.

Modern Highly-Active Anti-Retroviral Therapy (HAART) consists of various combinations of viral reverse transcriptase (RT), protease (Pro), and integrase (Int) inhibitors. Trimeris and Roche Pharmaceuticals also recently introduced a fusion inhibitor (Fuzeon), which prevents binding and fusion of infectious HIV particles to the plasma membrane of CD4⁺ T-lymphocytes, as a last-approach treatment against terminal AIDS associated with the appearance of HAART-resistant virus particles. In general, anti-HIV/AIDS therapies are prohibitively expensive—costing between $15,000-$30,000 per individual annually, and HAART frequently causes toxic clinical side-effects, including nausea, vomiting, diarrhea, as well as severe metabolic imbalances and liver/kidney dysfunction. During the course of anti-retroviral therapy, HAART-resistant particles may emerge through specific mutations conferring resistance to RT-nucleoside analog inhibitors (e.g., zidovudine; didanosine) or protease inhibitors (e.g., ritonavir; saquinavir). The appearance of these resistant particles may correlate with secondary viremias in HIV-infected patients, diminished CD4⁺ T-cell levels (<200 cells/μl), development of opportunistic infections (e.g., Toxoplasma, Pneumocystis, Cryptococcus, cytomegalovirus. Epstein Barr virus, etc.), and progression to terminal AIDS. Unfortunately, the majority of HIV-infected individuals (>25 million) in central and sub-Saharan Africa either cannot afford or do not have access to modern anti-HIV/AIDS drugs and, in many instances, are too isolated to reach medical treatment facilities.

A shortcoming of HAART is that, while these drugs target and inhibit the functions of various HIV proteins (RT, Pro, Int), none of the HAART-regimen compounds are designed to prevent viral gene expression and synthesis of HIV proteins (e.g., RT, Pro, Int, p24^{Gag}, gp120^{Env}) in infected cells.

A second limitation of HAART is the fact that the majority of HAART drugs do not penetrate the blood-brain- or blood-nerve-barriers (BBB; BNB). Thus, these drugs do not adequately address the neurological-involvement of HIV/AIDS. Indeed, the CNS is thought to serve as an immune-privileged reservoir for HIV-1 replication in which HAART-resistant virus particles can emerge to cause secondary viremia associated with terminal AIDS. Thus, there is an urgent need to identify new targets for the development of effective alternative anti-HIV/AIDS therapies.

Werner's syndrome is a homozygous recessive premature aging disease associated with shortened stature, cellular and physiological senescence, and a predisposition for development of certain malignancies, such as carcinomas, sarcomas, and melanomas. The Werner's syndrome helicase protein (WRN) contains 3-5′ exonuclease activity, ATPase activity, and seven helicase repeats, and is comprised of 1432 amino acid residues (FIG. 2. Moser M J et al., Nucleic Acids Res. 28:648-654; Moser M J et al., Nucleic Acids Res. 28:648-654; Suzuki N et al., Nucleic Acids Res. 27:2361-2368; Shen J et al., Mech. Ageing Dev. 122:921-944; Huang S et al., Nat. Genet. 20:114-116; Cooper M P et al., Genes Dev. 14:907-912). WRN shares significant amino acid sequence homologies with numerous DEAD-box RNA-helicases that function during transcriptional elongation and pre-mRNA-processing or splicing in different organisms (FIG. 2). Mutations of the WRN gene locus on chromosome 8 (p11-p12) typically result in the production of a highly-unstable truncated WRN protein, abrogate WRN helicase activity, and cause Werner's syndrome (Shen J et al., Mech. Ageing Dev. 122:921-944; Mohaghegh P et al., Int. J. Biochem. Cell Biol. 34:1496-1501; Bachrati C Z et al., Biochem. J. 374:577-606). The WRN helicase has been reported to interact with Ku70/Ku80 components of the DNA-dependent protein kinase (DNA-PK) complex and with the p53 tumor suppressor protein (Cooper M P et al., Genes Dev. 14:907-912; Orren D K et al., Nucleic Acids Res. 29:1926-1934; Li B et al., J. Biol. Chem. 275:28349-28352; Karmakar P et al., J. Biol. Chem. 277:18291-18302; Blander G et al., J. Biol. Chem. 274:29463-29469; Spillare E A et al., Genes Dev. 13:1355-1360; Nehlin J O et al., Ann. NY Acad. Sci. 908:167-179; Blander G et al., J. Biol. Chem. 277:50934-50940; Yang Q et al., J. Biol. Chem. 277:31980-31987). Interestingly, the WRN helicase is required for induction of the p53 transcriptional-response by genotoxic stress-inducing agents, such UV-irradiation or treatment of cells with adriamycin which causes G2/M cellular growth-arrest (Blander G et al., J. Biol. Chem. 274:29463-29469; Spillare E A et al., Genes Dev. 13:1355-1360; Blander G et al., J. Biol. Chem. 277:50934-50940; Yang Q et al., J. Biol. Chem. 277:31980-31987). These observations support an essential role for WRN during RNA Pol II-dependent transcriptional activation. WRN helicase activity is essential for RNA Pol II-dependent transcription (Balajee A S et al., Mol. Biol. Cell 10:2655-2668) and it is likely that WRN facilitates transcriptional elongation by unwinding RNA secondary structures that pose a barrier to the transcribing RNA Polymerase.

The Human Immunodeficiency Virus Type-1 (HIV-1) infects CD4⁺ T-lymphocytes and macrophages and causes Acquired Immune-Deficiency Syndrome (AIDS) and severe immune-suppression associated with loss of helper CD4⁺ T-cell functions. HIV-1 infects T-lymphocytes and macrophages within mucosal regions, such as intestinal Peyer's Patches, and enters the plasma through lymphatic or circulatory routes. Dendritic cells at primary infection sites facilitate the persistence of HIV-1 particles on mucosal membranes, increasing the probability for infection of susceptible immune cells (Geijtenbeek T B et al., Cell 100:587-597; Turville S G et al., Nat. Immunol. 3:975-983). Initial HIV-1-infection results in activation of a CD8⁺ cytotoxic T-lymphocyte (CTL) response, production of neutralizing antibodies, and lymphoadenopathies associated with T-cell and B-cell proliferation in germinal centers of lymph nodes. Cytopathic effects, resulting from virus replication and the production of inflammatory cytokines (IL-1β, TNF-α, TGF-β) or toxic reactive species (NO, ONOO⁻), cause progressive decline in the CD4⁺ T-lymphocyte population associated with immune-suppression and development of clinical AIDS (<200 CD4⁺ T-cells/μl). Immune-escape variants of HIV-1, resistant to neutralizing antibodies or cell-mediated immunity, invariably emerge with T-cell tropism (as compared to macrophage-tropic HIV-1 which predominates during early infections) to establish disseminated virus spread throughout lymphoid tissues and the CNS.

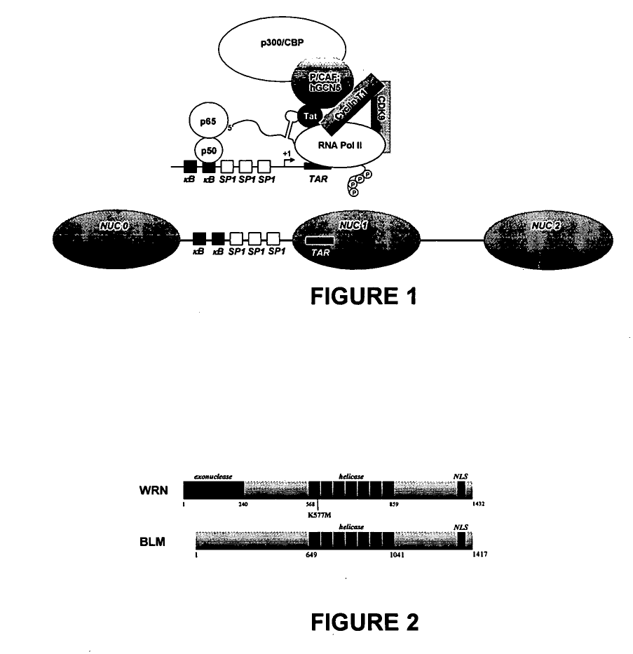

Following reverse transcription in the cytoplasm of HIV-infected cells, the double-stranded proviral DNA becomes integrated into the host's genome. Thus, transcription and expression of viral gene products (structural; regulatory proteins), as well as HIV-1 replication, are significantly controlled by cellular factors and their interactions with the viral transcriptional activator, Tat, in nuclei of infected cells. The Tat peptide is comprised of 81-101 amino acid residues encoded by two exons, and contains a nuclear localization signal (NLS), core domain, and an arginine-rich motif (ARM) that binds a uracil-containing bulge within a Tat-Activated Region-RNA (TAR-RNA) stem-loop structure (Kao S Y et al., Nature 330:489-493; Berkhout B. et al., Cell 59:273-282; Laspia M F et al., Cell 59:283-292; Marciniak R A et al., Cell 63:791-802). In absence of Tat, TAR-RNA suppresses transcriptional elongation by RNA Polymerase II and causes premature termination of viral mRNA synthesis. Tat functions as an anti-terminator and stimulates HIV-1 transcription and virus replication by countering TAR-RNA-mediated suppression, allowing RNA Polymerase II to clear the TAR-RNA sequence to produce full-length HIV-1 transcripts (Feinberg M B et al., PNAS 88:4045-4049; Keen N J et al., PNAS 93:2505-2510; Laspia M F et al., J. Mol. Biol. 232:732-746; Kato H et al., Genes Dev. 6:655-666; Marciniak R A et al., EMBO J. 10:4189-4196; Berkhout B et al., Cell 62:757-767). The Tat protein has also been shown to interact with cellular transcriptional coactivators/acetyltransferases, p300/CREB-binding protein (p300/CBP) and p300/CBP-associated factor (P/CAF); hGCN5, tethered on TAR-RNA (FIG. 1.). Tat is acetylated by p300/CBP on lysine residues, K50/K51, and by P/CAF on K28/K50 (Deng L et al., Virology 277:278-295; Dorr A et al., EMBO J. 21:2715-2723; Mujtaba S et al., Mol. Cell 9:575-586; Kiernan R E et al., EMBO J. 18:6106-6118; Col E et al., J. Biol. Chem. 276:28179-28184; Kaehlcke K et al., Mol. Cell 12:167-176). Acetylation of Tat on K28 by P/CAF stimulates interactions with the PTEF-b complex (containing human cyclin T1-cdk9) which is recruited by Tat to the viral promoter to phosphorylate heptaserines within the carboxyl-terminus of RNA Pol II to enhance elongation (Dorr A et al., EMBO J. 21:2715-2723; Kiernan R E et al., EMBO J. 18:6106-6118; Bres V et al., EMBO J. 21:6811-6819). Cellular transcription factors (e.g., NF-κB, SP1), assembled on upstream responsive-enhancer elements, synergize with Tat to drive activated HIV-1 transcription and replication (FIG. 1.). Interestingly, numerous studies have demonstrated that HIV-1^{ΔTat} proviruses, deleted for Tat sequences (HIV-1^{ΔTat}), retain significant replication competence suggestive that cellular factors can functionally substitute for Tat by countering TAR-RNA-suppressive effects upon RNA Pol II elongation to promote basal HIV-1 replication (Sadaie M R et al., New Biol. 2:479-486; Popik W et al., Virology 189:435-447; Sabino E et al., AIDS Res. Hum. Retroviruses 9:1265-1268; Verhoef K et al., Virology 237:228-236; Sabino E et al., AIDS 8:901-909). These factors are predicted to play important roles for continuous low-level viral gene expression and replication in HIV-1-infected tissue reservoirs (lymphoid; CNS) during long-term or chronic HIV-infections.

The present inventors, therefore, sought to identify cellular factors that substitute for Tat functions to promote basal HIV-1 transcription and replication. Based upon its ability to activate RNA Pol II-dependent transcription through an undefined mechanism (Balajee A S et al., Mol. Biol. Cell 10:2655-2668), an analysis of whether WRN helicase might transcriptionally-activate the HIV-1 LTR in the presence or absence of the viral Tat protein was undertaken. Surprisingly, through these studies it was discovered that the WRN helicase is an essential cofactor for HIV-1 transcription and viral replication. WRN trans-activates the HIV-1 LTR in absence of Tat, but synergizes with Tat to drive activated HIV-1 transcription, dependent upon WRN-associated helicase activity and intact TAR-RNA element. WRN^{-I-} fibroblasts, lacking a functional WRN helicase, are significantly impaired for basal and activated HIV-1 transcription and inhibition of WRN helicase functions through expression of the trans-dominant negative WRN_{K577M} mutant inhibits HIV-1 LTR trans-activation and markedly represses the intracellular synthesis of viral structural proteins (p24^{Gag}) in HIV-1-infected lymphocytes.

These findings identify the WRN helicase as a novel candidate target for the development of alternative small molecule anti-HIV/AIDS therapies. In addition, inhibition of WRN helicase functions or interactions with HIV components (Tat, TAR-RNA) potently represses basal and activated HIV-1 transcription and viral replication in transfected cells as well as in HIV-1-infected H9_{HIV-1IIIB} lymphocytes. These results suggest that small molecule inhibitors of WRN helicase that block its enzymatic functions or prevent WRN-interactions with HIV components, either alone or in combination with existing HAART-regimen compounds, could form a new class of anti-HIV/AIDS suppressants to inhibit virus replication which is not affected by modern HAART. These new anti-HIV/AIDS inhibitors would likely prove more effective towards reducing HIV-1 burden than existing anti-retroviral therapies.

The Tat protein binds the TAR-RNA stem-loop in HIV-1 transcripts to activate viral transcription by preventing the terminating effects of TAR-RNA secondary structure (Kao S Y et al., Nature 330:489-493; Berkhout B. et al., Cell 59:273-282; Laspia M F et al., Cell 59:283-292; Marciniak R A et al., Cell 63:791-802) and through the recruitment of PTEF-b (human cyclin T1-cdk9) complexes which phosphorylate heptaserines in the carboxyl-terminus of RNA Pol II (Wei P et al., Cell 92:451-462; Flores O et al., PNAS 96:7208-7213; Bieniasz P D et al., EMBO J. 17:7056-7065; Bieniasz P D et al., PNAS 96:7791-7796). However, numerous studies have demonstrated that HIV-1^{ΔTat} proviruses, deleted for Tat sequences, retain significant replication-competence suggesting that cellular factors contribute to basal HIV-1 replication by countering the suppressive effects of TAR-RNA and functionally substituting for Tat (Sadaie M R et al., New Biol. 2:479-486; Popik W et al., Virology 189:435-447; Sabino E et al., AIDS Res. Hum. Retroviruses 9:1265-1268; Verhoef K et al., Virology 237:228-236; Sabino E et al., AIDS 8:901-909). Through efforts to identify factors that support basal HIV transcription, it has been found that the WRN helicase is essential for HIV replication and that WRN significantly trans-activates the HIV-1 LTR, in absence of Tat, dependent upon TAR-RNA and WRN-associated helicase activity. WRN synergizes with Tat on TAR-RNA to drive activated viral transcription and HIV-1 replication and, importantly, WRN^{-I-} fibroblasts (lacking functional WRN) are significantly impaired for basal and activated HIV-1 transcription (Moser M J et al., Nucleic Acids Res. 28:648-654). Repression of WRN helicase activity, through expression of a trans-dominant-negative WRN_{K577M} mutant (Bai Y et al., Hum. Genet. 113:337-347), inhibits HIV-1 transcription and blocks intracellular synthesis of p24^{Gag} in HIV-infected lymphocytes.

Cellular factors that contribute to basal HIV-1 transcription and replication are likely to exert their effects upon the TAR-RNA secondary structure which serves as a potent barrier to RNA Pol II transcriptional elongation. Indeed, eukaryotic cells contain numerous proteins that enhance transcriptional elongation (e.g., TFIIF or RAP74; SPT5, PTEF-b). Balajee et al. (1999) demonstrated that the Werner's syndrome helicase (WRN) plays an important role during RNA Pol II-dependent transcription, as WRN-deficient cells (WRN^{-I-}) exhibit an overall reduction in RNA Pol II-synthesized transcripts (Balajee A S et al., Mol. Biol. Cell 10:2655-2668). Their study further showed that effects of WRN upon RNA Pol II-dependent transcription requires WRN-associated helicase activity as well as a 27-amino acid direct-repeat motif spanning residues 424-477 (Balajee A S et al., Mol. Biol. Cell 10:2655-2668). A single amino acid substitution mutation (K577M) within the WRN helicase domain abolishes the ability of WRN to support RNA Pol II-dependent transcription in vitro (Bai Y et al., Hum. Genet. 113:337-347; Balajee A S et al., Mol. Biol. Cell 10:2655-2668). Shiratori et al. (2002) reported that WRN enhances RNA Pol I-dependent transcription of ribosomal RNA genes (Shiratori M et al., Oncogene 21:2447-2454); and Gray et al. (1998) demonstrated that the WRN helicase localizes at transcriptionally active sites within nuclei of cycling cells (Gray M D et al., Exp. Cell. Res. 242:487-494). Ye et al. (1998) also reported transcriptional activation by WRN in yeast two-hybrid experiments (Ye L et al., Exp. Gerontol. 33:805-812). It remains unclear how WRN helicase activity contributes to cellular transcription and whether WRN exerts its functions at the level of transcriptional initiation or elongation. Suzuki et al. (1999) have shown RNA-specificity of WRN-associated helicase activity (Suzuki N et al., Nucleic Acids Res. 27:2361-2368) and, collectively, these results suggest that WRN may promote transcriptional activation through unwinding of suppressive RNA secondary structures.

### EXAMPLES

### Example 1

Amino acid sequence comparisons were performed using NIH/NCBI BLASTsearch of the Swissprot database for sequences GI:5739524 (human WRN protein) and GI:4557365 (human BLM protein). WRN and BLM helicases share significant amino acid sequence homologies with RNA-dependent helicases from different organisms (Table 1).

TABLE 1

| WRN Homologies | BLM Homologies |
|---|---|
| Yeast Pre-mRNA Processing | Murine RNA-Dependent Helicase |
| Helicase PRP5 | P68 |
| Human RNA-Dependent Helicase | Human RNA-Dependent Helicase |
| P72 | P68 |
| Yeast RNA-Dependent Helicase | Human RNA-Dependent Helicase |
| DBP10 | P72 |
| Murine RNA-Dependent Helicase | Xenopus ATP-Dependent RNA |
| PL10 | Helicase P54 |
| Human RNA-Dependent Helicase | Human ATP-Dependent RNA |
| P68 | Helicase P54 |
| Human ATP-Dependent RNA | Human ATP-Dependent RNA |
| Helicase P54 | Helicase DDX10 |
| Xenopus ATP-Dependent RNA | Human ATP-Dependent RNA |
| Helicase P54 | Helicase P47 |

BLM and WRN are not functionally similar with respect to their abilities to support RNA Pol II-dependent transcription, in agreement with dissimilar phenotypes and clinical symptoms linked to WRN or BLM deficiencies (Shen J et al., Mech. Ageing Dev. 122:921-944; Bachrati C Z et al., Biochem. J. 374:577-606; Nehlin J O et al., Ann. NY Acad. Sci. 908:167-179). WRN helicase activity is essential for RNA Pol II-dependent transcription and it is likely that WRN facilitates transcriptional elongation by unwinding RNA secondary structures that pose a barrier to the transcribing RNA Polymerase.

### Example 2

To determine whether the WRN or BLM helicases influence HIV-1 transcriptional activity and viral replication, HeLa cells were co-transfected with an HIV-1 LTR-luciferase reporter plasmid (0.5 μg), RSV-HIV-1 Tat (0.5 μg), and increasing amounts of CMV-WRN or CMV-BLM expression constructs (0.25, 0.5, 1.0 μg). Following transfection, the cells were lysed by repeated freeze-thawing and luciferase assays were performed using equivalent amounts of total cellular proteins.

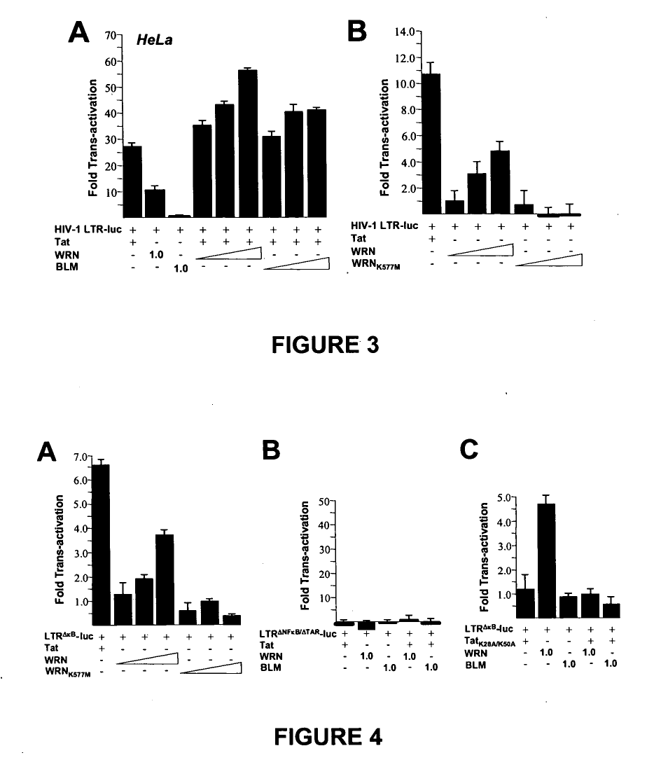

Results in FIG. 3A demonstrate that the viral trans-activator, Tat, significantly activates transcription (approximately 27-fold) from the HIV-1 LTR. The WRN helicase alone markedly trans-activates the HIV-1 LTR (approx. 10-fold) in absence of Tat, however, the BLM helicase does not affect basal HIV-1 LTR transcriptional activity (FIG. 3A). Surprisingly, co-expression of Tat with increasing amounts of CMV-WRN drastically increases Tat-mediated LTR trans-activation (nearly 60-fold), whereas BLM co-expression does not significantly influence Tat-mediated transcriptional activation (FIG. 3).

Next, an assay was performed to determine whether WRN-associated helicase activity is required for WRN-mediated trans-activation from the HIV-1 LTR. HeLa cells were co-transfected as in A with the HIV-1 LTR-luciferase reporter plasmid (0.5 μg), RSV-HIV-1 Tat (0.5 μg), or increasing amounts of CMV-WRN and CMV-WRN_{K577M} (a trans-dominant negative helicase-defective mutant) (0.25, 0.5, 1.0 μg) in the absence of HIV-1 Tat expression. Results shown in FIG. 3B are averages from duplicate assays and error bars representing standard deviations between samples are provided. As the results illustrate, the WRN helicase alone transcriptionally activates the HIV-1 LTR in a dose-dependent manner. The WRN_{K577M} mutant, however, fails to support LTR-driven transcription and represses basal transcription to levels below base-line (FIG. 3B). These results suggest that WRN-associated helicase activity is essential for WRN-mediated transcriptional activation from the HIV-1 LTR.

To determine whether the TAR-RNA stem-loop secondary structure is sufficient to support WRN-mediated LTR trans-activation, HeLa cells were co-transfected with an HIV-1 LTR^{ΔκB} luciferase mutant reporter construct, lacking upstream NF-κB-responsive enhancer sequences (transcription from this construct is principally regulated at the level of elongation by TAR-RNA), RSV-HIV-1 Tat, or increasing amounts of CMV-WRN or CMV-WRN_{K577M} (0.25, 0.5, 1.0 μg). Luciferase assays were performed as described using equivalent amounts of total cellular proteins. Results in FIG. 4A, demonstrate that Tat-mediated trans-activation from the HIV-1 LTR^{ΔκB}-luciferase reporter construct is markedly reduced (6.5-fold versus >20-fold) compared to the wild-type LTR. Surprisingly, the WRN helicase activated transcription from the HIV-1 LTR^{ΔκB}-luciferase construct, in a dose-dependent manner, to a level approximately one-half that observed for Tat (FIG. 4A). The WRN_{K577M} helicase-defective mutant did not significantly promote transcription from the HIV-1 LTR^{ΔκB}-luciferase reporter construct (FIG. 4A).

HeLa cells were co-transfected as in A with a double-mutant, HIV-1 LTR^{ΔNFκB/ΔTAR}-luciferase reporter plasmid, deleted for κB-responsive elements and TAR-RNA sequences, RSV-HIV-1 Tat, CMV-WRN, or CMV-BLM. Deletion of both the κB-responsive elements and the TAR-RNA region (HIV-1 LTR^{ΔNFκB/ΔTAR}-luciferase) completely abrogated transcriptional activation by HIV-1 Tat, as well as by the WRN helicase, indicative that these factors require an intact TAR-RNA secondary structure for their transcriptional activating effects (FIG. 4B).

The next experiment was undertaken to determine whether interactions with the Tat protein influence WRN's ability to trans-activate the HIV-1 LTR. HeLa cells were co-transfected with the HIV-1 LTR^{ΔκB}-luciferase, CMV-WRN or CMV-BLM, and a transcriptionally-inactive Tat mutant, Tat_{K28A/K50A}, defective for binding the TAR-RNA secondary structure (Kiernan R E et al., EMBO J. 18:6106-6118). All experiments were performed in duplicate or triplicate and error bars representative of standard deviations are shown in FIG. 4C. As shown, co-expression of the Tat_{K28A/K50A} mutant inhibited WRN-mediated trans-activation from the HIV-1 TAR-RNA. The BLM DNA-helicase had no significant influence upon basal HIV-1 LTR^{ΔκB} transcriptional activity (FIG. 4C). Since the Tat_{K28A/K50A} mutant protein is defective for binding HIV-1 TAR-RNA (Kiernan R E et al., EMBO J. 18:6106-6118), these results suggest that WRN directly interacts with the viral trans-activator on the HIV-1 LTR -possibly to promote “recycling” of Tat through dissociation of Tat/TAR-RNA complexes by unwinding TAR-RNA.

### Example 3

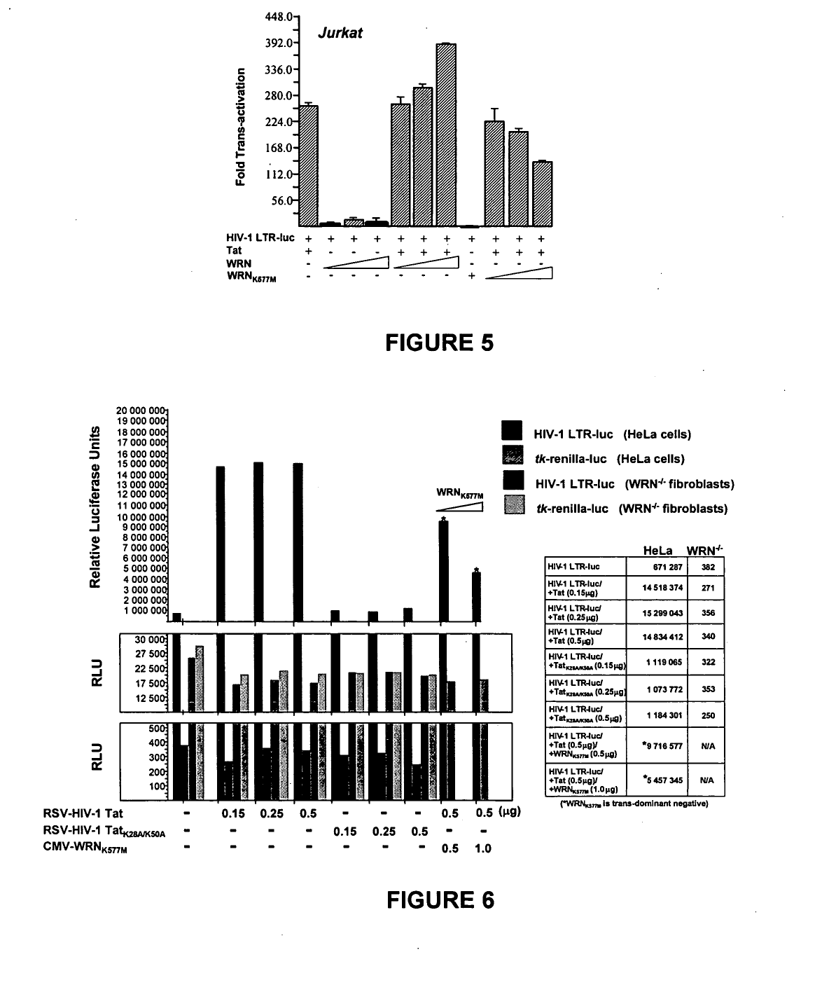

To determine the influence of the WRN helicase upon HIV-1 LTR transcriptional activation in CD4⁺ lymphocytes, Jurkat E6.1 T-cells were co-transfected with HIV-1 LTR-luciferase (0.5 μg), RSV-HIV-1 Tat (0.5 μg), and increasing amounts of CMV-WRN or CMV-WRN_{K577M}, which expresses a trans-dominant negative helicase-defective mutant (0.25, 0.5, 1.0 μg). Following transfection, the cells were lysed by repeated freeze-thawing and luciferase assays were performed, in duplicate, using equivalent amounts of total cellular proteins. Results shown in FIG. 5 demonstrate that Tat potently activates transcription (approximately 250-fold) from the HIV-1 LTR in Jurkat E6.1 lymphocytes. Co-expression of increasing amounts of the WRN helicase results in significantly higher-levels of trans-activation by Tat (approximately 395-fold. FIG. 5). Inhibition of endogenous WRN helicase functions, through co-expression of increasing amounts of the trans-dominant-negative WRN_{K577M} mutant, repressed nearly one-half of Tat's transcriptional activation from the HIV-1 LTR in transfected Jurkat E6.1 lymphocytes (FIG. 5). These results indicate that WRN is an important cofactor for HIV-1 LTR trans-activation and suggest that inhibition of the WRN helicase or its interactions with Tat/TAR-RNA complexes may be a plausible approach to prevent HIV-1 replication in infected individuals.

### Example 4

In order to determine whether the WRN helicase is an essential cofactor for HIV-1 transcription and virus replication, basal and Tat-activated LTR-driven transcription were measured in WRN^{-I-}fibroblasts. These fibroblasts were obtained from Werner's syndrome patients and lack a functional WRN helicase protein (Moser M J et al., Nucleic Acids Res. 28:648-654). Others have reported that Werner's syndrome patient fibroblasts possess a general impairment in RNA Pol II-dependent transcriptional activation (Balajee A S et al., Mol. Biol. Cell 10:2655-2668).

Therefore, we co-transfected immortalized WRN^{-I-} fibroblasts with a tk-renilla luciferase reporter plasmid (which produces red luminescence as a result of renilla luciferase expression driven by the Herpes Simplex Virus thymidine kinase (tk) minimal gene promoter)(0.5 μg) and an HIV-1 LTR-luciferase reporter construct (which produces green luminescence as a result of firefly luciferase expression driven by the HIV-1 LTR) (0.5 μg), and increasing amounts of RSV-HIV-1 Tat or RSV-HIV-1 Tat_{K28A/K50A} (0.15, 0.25, 0.5 μg) in the absence or presence of the trans-dominant negative CMV-WRN_{K577M} mutant (0.5, 1.0 μg). The CMV-WRN_{K577M} trans-dominant-negative helicase-defective mutant was co-expressed in certain experiments to inhibit Tat-activated HIV-1 LTR transcription and endogenous WRN functions in HeLa cells (Bai Y et al., Hum. Genet. 113:337-347). Samples were normalized to yield approximately equivalent renilla-luciferase expression to control for transfection efficiencies and any differences in overall transcription levels between WRN^{-I-} fibroblasts and HeLa cells. Normalized extracts were then compared to determine basal-level and Tat-activated HIV-1 LTR-luciferase transcription. Relative luciferase measurements from a representative experiment are shown in the table at right.

The basal HIV-1 LTR-driven luciferase activity in transfected HeLa extracts was 671,287 relative luciferase units (RLUs), compared to 382 RLUs in transfected WRN^{-I-} fibroblasts which lack the WRN helicase (FIG. 6). Importantly, Tat-activated HIV-1 LTR transcription was severely repressed in WRN^{-I-} fibroblasts, which exhibited only 340 RLUs (0-fold trans-activation) at the highest concentration of RSV-HIV-1 Tat-compared to 14,834,412 RLUs (22-fold trans-activation) in HeLa cells (FIG. 6). The tk-renilla-luciferase transcription levels were similar in WRN^{-I-} fibroblasts and HeLa cells indicative of comparable transfection efficiencies in both cell-types (FIG. 6). These results support our previous findings that the WRN helicase plays an essential role for basal and Tat-activated HIV-1 LTR transcription in transfected HeLa cells and Jurkat E6.1 lymphocytes. Moreover, the helicase-defective, trans-dominant-negative WRN_{K577M} mutant (Bai Y et al., Hum. Genet. 113:337-347) significantly repressed Tat-activated HIV-1 LTR transcription in HeLa cells (5,457,345 RLUs at the highest concentration of WRN_{K577M} versus 14,518,374 RLUs for Tat alone), suggestive that trans-inhibition of endogenous WRN functions may be a plausible approach for repression of HIV-1 transcription and virus replication (FIG. 6). These results, derived from experiments using immortalized human fibroblasts lacking a functional WRN helicase (Moser M J et al., Nucleic Acids Res. 28:648-654), demonstrate that WRN is essential for basal and activated HIV-1 LTR transcription.

### Example 5

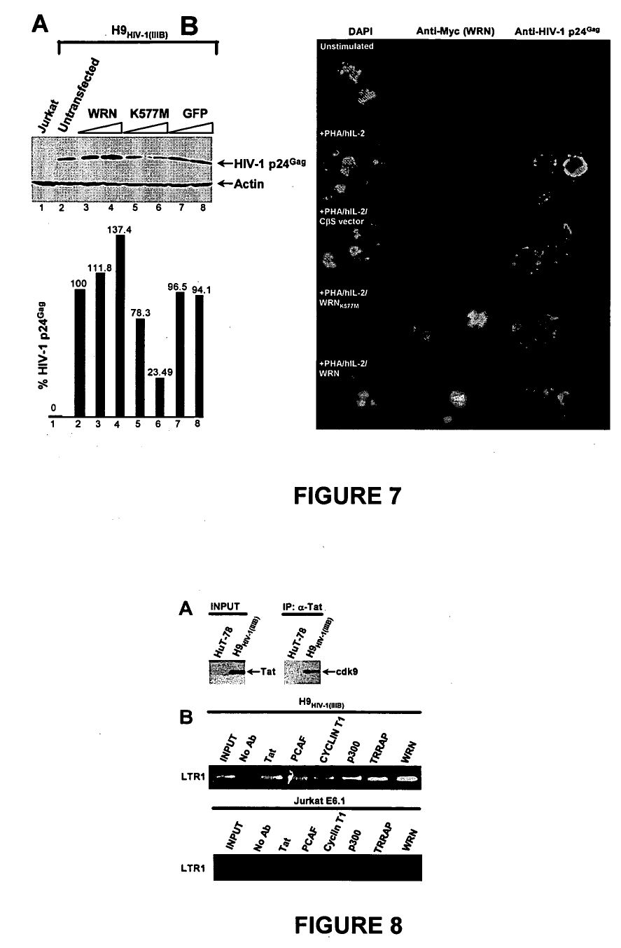

The influence of over-expressing wild-type WRN, or trans-dominant-negative WRN_{K577M}, proteins upon HIV-1 replication and the production of intracellular viral structural components was tested in HIV-infected H9 lymphocytes that were stimulated with PHA (10 ng/ml) and human IL-2 (50 U/ml) . HIV-1_{IIIB}-infected H9 lymphocytes were transfected with increasing amounts of CMV-WRN, CMV-WRN_{K577M}, or CMV-green fluorescent protein (GFP) negative control (1.0, 3.0 μg) and then stimulated with PHA (10 ng/ml) and hIL-2 (50 U/ml) to induce HIV-1 replication. The HIV-1 p24^{Gag} major capsid and cellular actin proteins were detected by immuno-blotting using appropriate primary antibodies. Protein expression was quantified using a densitometer and is represented graphically below immunoblotting results shown in FIG. 7A.

These immunoblotting results demonstrate that over-expression of wild-type WRN results in significantly higher intracellular production of the p24^{Gag} major viral capsid protein and increased HIV-1 replication in transfected HIV-1-infected H9 lymphocytes (percentages of intracellular HIV-1 p24^{Gag} are represented graphically), whereas, ectopic over-expression of the trans-dominant-negative WRN_{K577M} mutant represses production of the p24^{Gag} major capsid protein and inhibits HIV-1 replication (FIG. 7A). The GFP negative control had no effect upon p24^{Gag} expression and viral replication in stimulated HIV-infected H9 lymphocytes. Actin expression was similar for all cellular extracts and is shown as a protein-loading control (FIG. 7A).

Immunofluorescence-microscopy was next performed to directly assess whether over-expression of Myc-epitope-tagged WRN or the trans-dominant-negative WRN_{K577M} mutant influence HIV-replication and production of p24^{Gag} in infected H9_{HIV-1IIIB} lymphocytes. HIV-1-infected H9 lymphocytes were transfected with increasing amounts of CMV-WRN, CMV-WRN_{K577M}, or CβF empty vector (0.12, 0.25 μg) and were stimulated with PHA and hIL-2 as described above. Immunofluorescence-microscopy was performed to determine the influence of WRN or WRN_{K577M} over-expression upon production of HIV-1 p24^{Gag} and viral replication. Expression of Myc-epitope-tagged WRN and WRN_{K577M} proteins in transfected H9 cells was detected using a rabbit anti-Myc polyclonal antibody (Moser M J et al., Nucleic Acids Res. 28:648-654). Production of intracellular HIV-1 p24^{Gag} capsid protein was detected using a monoclonal anti-HIV-1 p24^{Gag} antibody. DAPI-staining was performed to visualize nuclei.

As shown in FIG. 7B, PHA/hIL-2-stimulation significantly induces HIV-1 gene expression and p24^{Gag} production in infected H9 lymphocytes. Over-expression of WRN-Myc results in increased intracellular p24^{Gag} compared to surrounding untransfected cells or the C□F empty vector (FIG. 7B). Moreover, HIV-infected H9 cells, expressing the trans-dominant-negative WRN_{K577M}-Myc protein, exhibit markedly reduced levels of p24^{Gag} indicative that interference with WRN negatively affects HIV-1 replication (FIG. 7B). These results indicate that WRN functions or interactions with viral components modulate HIV-1 replication in infected lymphocytes and demonstrate that trans-inhibition of the WRN helicase prevents intracellular synthesis of HIV-1 structural components (p24^{Gag}) and virus replication.

The HIV-1 Tat protein interacts with transcriptional coactivators, such as cyclin T1-cdk9, in HIV-infected cells to promote viral transcription and replication (FIG. 8A). The cyclin T1-cdk9 complex co-immunoprecipitates with Tat in HIV-1-infected H9 lymphocytes. Expression of Tat in H9_{HIV-1IIIB} cells is shown compared to uninfected HuT-78 control lymphocytes. A rabbit polyclonal anti-HIV-1 Tat antibody and protein G-agarose was used to immunoprecipitate Tat; and bound cdk9 was detected by immunoblotting.

To determine whether the WRN helicase is specifically recruited to transcriptional promoter complexes assembled on the HIV-1 LTR, chromatin-immunoprecipitation (ChIP) experiments were performed on HIV-1_{IIIB}-infected H9 lymphocytes that were stimulated with PHA (10 ng/ml) and hIL-2 (50 U/ml) to induce virus replication and on uninfected Jurkat E6.1 lymphocytes as a negative control.

Nucleoprotein transcription complexes bound to the HIV-1 promoter were cross-linked in vivo by treating cells with formaldehyde for 10 min. The cells were harvested by centrifugation at 4° C., washed twice with PBS, and chromatinized total genomic DNA was extracted by resuspending HIV-1-infected H9 lymphocytes or Jurkat E6.1 lymphocytes in SDS-lysis buffer (Upstate Biotechnology). Oligonucleosomal DNA fragments containing bound cross-linked protein complexes were generated by briefly sonicating samples at 4° C.

Antibodies that recognize HIV-1 Tat, PCAF, p300, Cyclin T1, WRN or TRRAP (p434) were added and protein-A-agarose was used to precipitate immune-complexes bound to oligonucleosomal DNA fragments. To identify factors specifically interacting on the HIV-1 LTR in the vicinity of TAR-RNA, oligonucleotide DNA primers (LTR1 forward 5′-ACTTTTCCGGGGAGGCGCGATC-3′ (SEQ ID NO:1); LTR1 reverse 5′-GCCACTGCTAGAGATTT-CCACACTG-3′ (SEQ ID NO:2)) annealing at positions −92 and +180 (Zhou M et al., PNAS 100:12666-12671) were used to amplify HIV-1 LTR fragments precipitated with various factors (HIV-1 Tat, WRN, TRRAP, PCAF, p300, Cyclin T1) by polymerase chain reaction (PCR); amplified PCR products were resolved by electrophoresis through a 2% TAE agarose gel and visualized by ethidium bromide-staining. As shown in FIG. 8B, HIV-1 Tat and the transcriptional coactivators, PCAF, p300, TRRAP, and Cyclin T1, were present bound to the HIV-1 LTR and the WRN helicase was strongly detected on the HIV-1 LTR in Tat/TAR-RNA complexes (FIG. 8B). No signal was amplified using the LTR1 primers, specific for HIV-1 LTR sequences, in ChIP reactions using uninfected Jurkat E6.1 lymphocytes as negative control. Importantly, these results indicate that the WRN helicase interacts in transcription complexes with Tat, PCAF, p300, Cyclin T1, and TRRAP recruited to the HIV-1 LTR—consistent with its essential role for viral transcription and replication in infected T-cells.

### Example 6

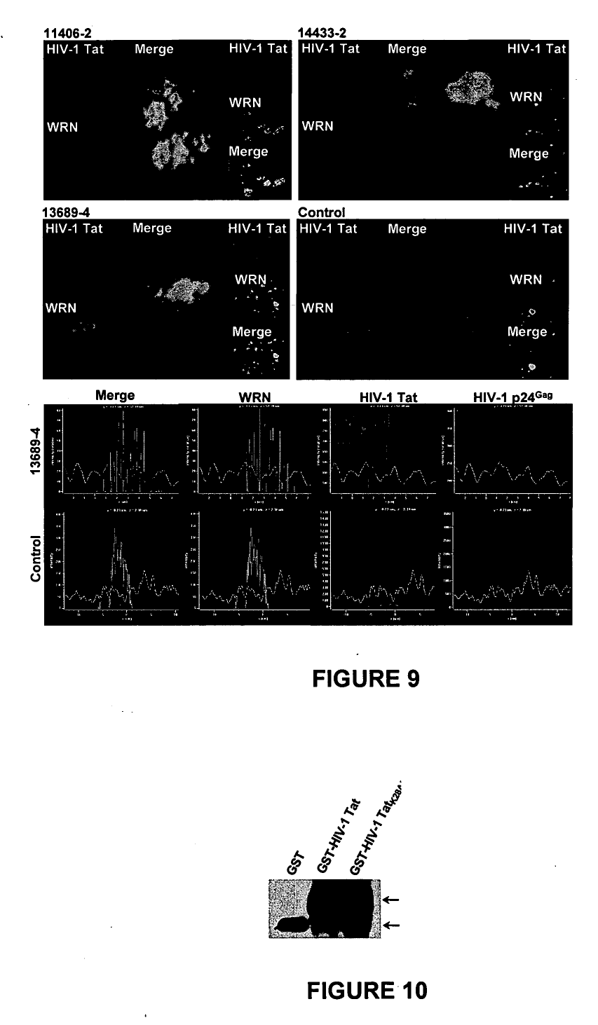

Finally, immunofluorescence-confocal laser scanning microscopy was (iCLSM) performed to determine whether the WRN helicase co-localizes with the viral trans-activator protein, Tat, in HIV-1-infected cells. Post-mortem HIV-infected CNS-tissues (cingulum; thalamus) obtained from three donor Neuro-AIDS patients diagnosed with primary HIV encephalopathies was analyzed. Primary anti-HIV-1 Tat (Advanced Bioscience Laboratories) and anti-WRN (Santa Cruz Biotechnology) antibodies and appropriate fluorescent secondary antibodies (FITC and Rhodamine Red-conjugated) (Jackson Immunoresearch Laboratories) were used to visualize co-localization between HIV-1 Tat and the WRN helicase in HIV-infected CNS tissues. The HIV-1 p24^{Gag} protein was detected using a monoclonal antibody. Three-dimensional composite images showing nuclear co-localization between Tat-WRN are shown in FIG. 9. The HIV-1 Tat protein (red) is expressed throughout the nucleoplasm and strongly co-localizes with the WRN helicase (green). Wide-field micrographs demonstrating co-localization between Tat-WRN in nuclei of HIV-infected cells in CNS-tissues are also shown. No non-specific HIV-1 Tat-staining was observed in uninfected control brain-tissue as an antibody control (FIG. 9). Quantification of relative fluorescence-intensities of HIV-1 Tat, HIV-1 p24^{Gag}, and WRN are shown for a representative Neuro-AIDS patient sample and uninfected CNS-tissue. Our findings suggest that the WRN helicase and Tat interact to synergistically drive viral transcription and replication in HIV-1-infected cells.

### Example 7

The WRN helicase may catalyze unwinding of TAR-RNA and dissociation of Tat/TAR-RNA complexes to promote transcriptional activation by “recycling” the viral trans-activator—allowing it to participate in multiple trans-activation events. The interactions between WRN and Tat, which may contribute to HIV-1 trans-activation and viral replication, may be biochemically characterized.

Radiolabeled TAR-RNA may be complexed with purified recombinant Tat protein and mixed with nuclear extracts, over-expressing wild-type WRN or extracts in which WRN functions are inhibited as a result of the trans-dominant negative WRN_{K577M} mutant (Bai Y et al., Hum. Genet. 113:337-347). Alternatively, nuclear extracts prepared from WRN^{-I-} fibroblasts, lacking WRN helicase activity (Moser M J et al., Nucleic Acids Res. 28:648-654), may be used in these experiments. TAR-RNA electrophoretic gel-mobility shift assays (EMSAs) may be performed to determine whether the WRN helicase unwinds the TAR-RNA stem-loop structure to dissociate Tat/TAR-RNA complexes promoting “recycling” of the Tat protein. Pair-wise alanine (Ala-Ala)-substitution mutants of HIV-1 Tat, defective for interactions with WRN, may be generated by PCR linker-scanning mutagenesis and tested for TAR-RNA-binding and “recycling” in vitro, as well as for their abilities to trans-activate the HIV-1 LTR and promote replication of HIV-1^{ΔTat} proviruses in transfected cells (Sadaie M R et al., New Biol. 2:479-486;. Popik W et al., Virology 189:435-447). Effects of specific Tat-mutations upon virus replication and infectivity may be determined by quantifying intracellular and extracellular p24^{Gag} levels.

The instant disclosure shows that the WRN helicase synergistically trans-activates the HIV-1 LTR with Tat, dependent upon TAR-RNA and WRN-associated helicase activity (see FIGS. 3; 4). The WRN helicase co-localizes with Tat in HIV-1-infected CNS-tissues from donor Neuro-AIDS patients diagnosed with primary encephalopathies. Moreover, a TAR-binding-defective Tat_{K28A/K50A} mutant (Kiernan R E et al., EMBO J. 18:6106-6118) inhibits the ability of WRN to support basal transcription from the HIV-1 LTR (see FIG. 4C) suggestive that Tat directly recruits WRN to the HIV-1 LTR to promote transcriptional activation. In addition, WRN helicase activity is essential for basal and Tat-activated LTR transcription, as WRN^{-I-} fibroblasts (Moser M J et al., Nucleic Acids Res. 28:648-654) do not support HIV-1 LTR-driven transcription (see FIG. 6); and expression of the trans-dominant-negative WRN_{K577M} mutant (Bai Y et al., Hum. Genet. 113:337-347) inhibits intracellular production of p24^{Gag} and HIV-1 replication in infected H9_{HIV-1IIIB} lymphocytes (see FIGS. 7A and 7B). The WRN helicase functions as a general activator of RNA Pol II-dependent transcription (Balajee A S et al., Mol. Biol. Cell 10:2655-2668), although its mechanism remains undefined. Gray et al. (1998) have shown that WRN localizes in transcriptionally-active sites in nuclei of cycling cells (Gray M D et al., Exp. Cell. Res. 242:487-494). Without being limited to any particular model or mode of action, the data of Examples 2-6 suggest that WRN functions as an anti-terminator by unwinding RNA-secondary structures that form during elongation, thereby preventing premature termination by RNA Pol II. Thus, novel anti-HIV/AIDS therapies to prevent viral replication may be identified and developed by elucidating the mechanism through which WRN promotes basal and Tat-activated HIV-1 LTR transcription.

Initially, a determination may be made whether WRN-associated helicase activity dissociates HIV-1 Tat/TAR-RNA complexes to promote “recycling” of the viral trans-activator. The TAR-RNA sequence may be subcloned and in vitro transcribed using T7 polymerase in the presence of α-[³²P]-UTP to generate a radioactively-labeled [³²P]-TAR-RNA riboprobe. Unincorporated radiolabel may be removed by electrophoresis and purification of the TAR-RNA oligonucleotide from a non-denaturing 7% TBE acrylamide gel. Recombinant GST-HIV-1 Tat may be incubated with the TAR-RNA probe and RNA electrophoretic gel-mobility shift assays (EMSAS) may be performed as described in Kiernan et al. (Kiernan R E et al., EMBO J. 18:6106-6118) by electrophoresing samples through a 5% non-denaturing TBE acrylamide gel to visualize HIV-1 Tat/TAR-RNA complexes (Kiernan R E et al., EMBO J. 18:6106-6118). Autoradiography may be performed to detect HIV-1 Tat-bound and unbound TAR-RNA; quantification may be performed using a densitometer. To determine the influence of the WRN helicase upon Tat/TAR-RNA complexes, nuclear extracts from HeLa cells transfected either with increasing amounts of CMV-WRN or CMV-WRN_{K577M} may be mixed and incubated with RNA EMSA reactions. Alternatively, nuclear extracts prepared from WRN^{-I-} fibroblasts, which lack functional WRN helicase (Moser M J et al., Nucleic Acids Res. 28:648-654), may be used. If the WRN helicase unwinds the TAR-RNA stem-loop secondary structure to promote dissociation of Tat/TAR-RNA, then incubation of binding reactions with nuclear extracts over-expressing WRN should result in significantly decreased formation of detectable Tat/TAR-RNA complexes and a corresponding increase in free TAR-RNA probe. Since WRN-mediated trans-activation requires WRN-associated helicase activity, addition of nuclear extracts prepared from transfected HeLa cells over-expressing the trans-dominant WRN_{K577M} helicase-inactive mutant (or nuclear extracts prepared from WRN^{-I-} fibroblasts (Bai Y et al., Hum. Genet. 113:337-347)) to reactions would likely result in increased Tat/TAR-RNA complex formation and diminished levels of the TAR-RNA free probe in RNA EMSAs.

To identify specific amino acid residues within HIV-1 Tat that interact with the WRN helicase, alanine (Ala)-substitution linker-scanning mutagenesis may be performed by creating pair-wise Ala-Ala substitutions at various positions within the HIV-1 Tat coding sequence in combination with an engineered restriction endonuclease cleavage site. Approximately eight linker-scanning (Ala-Ala) substitution mutants may be generated spanning the Tat protein. These mutants may be sequenced and subcloned into a pGEX expression vector, induced, and GST-HIV-1 Tat-derived mutant proteins may be purified from bacteria using Glutathione-Sepharose 4B (Amersham-Pharmacia Biotech).

Glutathione-S-transferase, GST-HIV-1 Tat, and GST-HIV-1 Tat_{K28A/K50A} recombinant proteins (Kiernan R E et al., EMBO J. 18:6106-6118) have been expressed and purified (FIG. 10). pGEX-based plasmids were transformed into E. coli, strain DH5α, and GST-fusion proteins were induced by treating cultures with IPTG (100 μM) for 2 hr. Bacterial cells were harvested, lysed by sonication, and recombinant proteins were bound to Glutathione-Sepharose 4B (Amersham-Pharmacia Biotech) overnight at 4° C. Matrices were washed and GST-proteins were eluted with 10 mM reduced glutathione buffer. Dialyzed purified protein fractions were stored at −80° C. in 200□1 aliquots.

The HIV-1 Tat-derived Ala-Ala-substitution mutant proteins may be assayed for TAR-RNA-binding in vitro and may be used to identify mutations that prevent recruitment of WRN to Tat/TAR-RNA complexes in RNA EMFSA experiments using a radiolabeled TAR-RNA probe. These mutants will also be directly used in GST-pull-down assays using HeLa nuclear extracts to identify mutations that abrogate interactions between HIV-1 Tat and the WRN helicase. Tat-derived Ala-Ala-substitution mutants, identified to be defective for binding WRN, but not TAR-RNA, may be subcloned into an RSV promoter-driven eukaryotic expression construct and co-transfected with an HIV-1 LTR-luciferase reporter plasmid in Jurkat E6.1 lymphocytes to determine their capacities to transcriptionally activate the HIV-1 LTR. Finally, in order to determine the importance of Tat-WRN interactions for HIV-1 replication, cell-lines containing HIV-1^{ΔTat} proviruses (Sadaie M R et al., New Biol. 2:479-486; Popik W et al., Virology 189:435-447), deleted for Tat sequences, may be transfected with RSV-constructs expressing various WRN-binding-defective Tat mutants. The relative intracellular production of p24^{Gag}, reflecting HIV-1 LTR transcriptional activity, may be determined by immunoblotting and the amounts of virus particles released into supernatants may be quantified by measuring extracelluar p24^{Gag} by ELISAs. These experiments will provide valuable insight regarding the role of Tat-WRN interactions for dissociation of Tat/TAR-RNA complexes and HIV-1 LTR trans-activation and virus replication.

### Example 8

Chromatin-immunoprecipitations (ChIPs) may be performed to detect the presence of cellular factors and their interactions with Tat/TAR-RNA, as well as interactions on upstream enhancer elements (κB, SP1). To address these questions, a biotinylated TAR-RNA probe may be incubated with nuclear extracts expressing HIV-1 Tat or various Tat-mutants defective for binding WRN. Alternatively, extracts in which WRN functions are inhibited (through WRN_{K577M} expression) or extracts prepared from WRN^{-I-} fibroblasts may be used in certain experiments. Nucleoprotein complexes assembled on TAR-RNA may be cross-linked using formaldehyde and precipitated with streptavidin-agarose. Effects of WRN interactions with Tat/TAR-RNA upon phosphorylation of RNA Polymerase II may be determined using phospho-RNA Pol II-specific antibodies in ChIP analyses. This influence of immune/cytokine-signaling (IL-2, TNF-α, TGF-β) upon recruitment of the WRN helicase to the HIV-1 promoter may also be determined.

The influence of WRN/Tat/TAR-RNA interactions upon recruitment of transcriptional coactivators, such as PCAF, p300, TRRAP and Cyclin T1-cdk9, to the HIV-1 LTR and their effects upon chromatin-modifications (histone H3/H4-acetylation) and RNA Pol II-phosphorylation may be determined. The effect of immune-signaling (IL-2, TNF-α, TGF-β) on recruitment of the WRN helicase by Tat to the HIV-1 LTR to promote trans-activation and viral replication may be determined.

The HIV-1 trans-activator recruits the transcriptional coactivators, p300/CBP and PCAF;hGCN5, to the viral LTR to promote transcriptional activation. Both p300/CBP and PCAF;hGCN5 have been demonstrated to acetylate the HIV-1 Tat protein. Acetylation of Tat on lysine residue K28A enhances interactions between Tat and the PTEF-b complex, containing Cyclin T1-cdk9 (Kiernan R E et al., EMBO J. 18:6106-6118; Bres V et al., EMBO J. 21:6811-6819). Recruitment of PTEF-b to Tat/TAR-RNA promotes hyper-phosphorylation of heptaserines within the carboxyl-terminus of RNA Pol II and facilitates synthesis of full-length HIV-1 transcripts (Wei P et al., Cell 92:451-462; Flores O et al., PNAS 96:7208-7213; Bieniasz P D et al., EMBO J. 17:7056-7065; Bieniasz P D et al., PNAS 96:7791-7796; Kiernan R E et al., EMBO J. 18:6106-6118; Bres V et al., EMBO J. 21:6811-6819). Zhou et al. (2003) recently demonstrated that Tat/TAR-RNA-dependent phosphorylation of RNA Pol II also stimulates co-transcriptional capping of viral mRNAs (Zhou M et al., PNAS 100:12666-12671). Using ChIP analyses, the data disclosed herein shows that the WRN helicase interacts in Tat-containing promoter complexes in the vicinity of the TAR-RNA element and trans-activates the HIV-1 LTR, dependent upon WRN-associated helicase activity and TAR-RNA. As lysine residues, K50/K51, within the ARM of Tat, which are targeted for acetylation by p300/CBP and PCAF; hGCN5 (Deng L et al., Virology 277:278-295; Dorr A et al., EMBO J. 21:2715-2723; Mujtaba S et al., Mol. Cell 9:575-586; Kaehlcke K et al., Mol. Cell 12:167-176; Col E et al., J. Biol. Chem. 276:28179-28184; Kiernan R E et al., EMBO J. 18:6106-6118; Bres V et al., EMBO J. 21:6811-6819), are known to directly bind phosphodiesters in the TAR-RNA stem-loop (Puglisi J D et al., Science 257:76-80; Puglisi J D et al., PNAS 90:3680-3684), it is possible that unwinding of TAR-RNA and dissociation of Tat/TAR-RNA complexes by WRN modulates interactions with the transcriptional coactivators, p300/CBP, PCAF;hGCN5, and PTEF-b. Recruitment of WRN through binding to Tat, therefore, may influence chromatin-modifications surrounding the HIV-1 LTR and phosphorylation of RNA Pol II on the viral promoter. Further, as inflammatory signals, TNF-α, TGF-β, IL-2, significantly trans-activate the HIV-1 LTR, promoting viral replication in latent tissue reservoirs, immune-receptor-signaling may influence interactions between Tat/WRN or other transcriptional coactivators recruited to the HIV-1 promoter. Therefore, the following experiments may be performed to determine the influence of TNF-α, TGF-β, and IL-2 upon Tat/WRN complexes as well as WRN-mediated transcriptional activation of the HIV-1 LTR.

To determine the influence of Tat/WRN interactions upon recruitment of transcriptional coactivators, p300/CBP, PCAF, hGCN5, TRRAP, and Cyclin T1-cdk9, to the HIV-1 LTR, ChIP analyses on HIV-1-infected H9_{HIV-1IIIB} lymphocytes may be performed as described in Example 5 using the LTR1 PCR primer-pair which amplifies nucleotides −92 to 180 surrounding the TAR-RNA region (Zhou M et al., PNAS 100:12666-12671). WRN functions may be inhibited by transfecting HIV-1-infected H9_{HIV-1IIIB} lymphocytes with increasing amounts of CMV-WRN_{K577M}, expressing the trans-dominant-negative WRN_{K577M} mutant (Bai Y et al., Hum. Genet. 113:337-347), and transfected cultures may be stimulated with PHA (10 ng/ml) and hIL-2 (50 U/ml) to induce HIV-1 replication. Intracellular and extracellular p24^{Gag} capsid protein levels may be measured by ELISAs and immuno-blotting as in Example 5 to quantify virus production. H9_{HIV-1IIIB} Cultures may be treated with formaldehyde to cross-link nucleoprotein complexes assembled on the HIV-1 LTR in vivo. The transfected H9_{HIV-1IIIB} lymphocytes may be harvested by centrifugation and chromatinized genomic DNA may be extracted using SDS-lysis buffer (Upstate Biotechnology). Oligonucleosomal DNA fragments may be generated by briefly sonicating the samples over ice and antibodies that specifically bind factors recruited to the HIV-1 LTR (e.g., Tat, PCAF, p300/CBP, TRRAP, Cyclin T1-cdk9, WRN) and protein A-agarose may be added to ChIP reactions followed by incubation overnight with agitation at 4° C. (see FIG. 8B). As demonstrated herein, inhibition of WRN functions, as a result of expressing the trans-dominant-negative WRN_{K577M} mutant, inhibits the HIV-1 LTR and markedly prevents the intracellular production of p24^{Gag} capsid protein (FIGS. 6 and 7). Thus, trans-inhibition of WRN functions may negatively affect the recruitment of essential transcriptional accessory factors, such as p300/CBP, PCAF;hGCN5, TRRAP, and Cyclin T1-cdk9, to Tat/TAR-RNA complexes.

ChIP analyses, using antibodies that immunoprecipitate these factors, may be performed to determine the effects of inhibiting WRN helicase functions upon Tat-mediated assembly of chromatin-remodeling complexes on the HIV-1 LTR. The influence of WRN-recruitment upon histone H3/H4-acetylation in the vicinity of the TAR-RNA element by ChIPs may also be investigated. Moreover, transcription factors (NF-κB, SP-1) bound to upstream enhancer elements within the HIV-1 LTR synergistically trans-activate viral gene expression, together with Tat/TAR-RNA complexes (Perkins N D et al., EMBO J. 12:3551-3558; Griffin G E et al., Nature 339:70-73; Parrott C et al., J. Virol. 65:1414-1419; Berkhout B et al., Cell 62:757-767; Schmid R M et al., Nature 352:733-736), and it is likely that dynamic interactions occur between Tat-associated factors on TAR-RNA and upstream NF-κB or SP-1-responsive elements.

Antibodies that recognize NF-κB (p65^{RelA}, p50, p52) and SP-1, may be used to determine whether inhibition of WRN functions, through expression of the trans-dominant-negative WRN_{K577M} mutant, alters the recruitment of upstream transcription components and synergism with WRN/Tat/TAR-RNA complexes (see FIG. 8B). The LTR1 primer-pair (Nuc 1) may be used to investigate histone H3/H4-acetylation surrounding the TAR-RNA region in ChIPs. In addition, PCR primers that amplify the Nuc 0 and Nuc 2 regions by ChIPs may be used to analyze chromatin-modifications within these regions of the HIV-1 LTR (see FIG. 1). Alternatively, to determine the effects of inhibiting WRN helicase functions upon recruitment of Tat-associated transcriptional coactivators (PCAF;hGCN5, p300/CBP, TRRAP, Cyclin T1-cdk9) to HIV-1 TAR-RNA, a synthetic biotinylated TAR-RNA riboprobe may be incubated with nuclear extracts prepared from PHA/hIL-2-stimulated HIV-1-infected H9_{HIV-1IIIB} lymphocytes that were either transfected with increasing amounts of CMV-WRN_{K577M}, CMV-WRN, or an empty CSF vector control. Streptavidin-agarose may be incubated with the samples at 4° C. and TAR-RNA-bound nucleoprotein complexes may be precipitated by centrifugation. Bound transcription factors may be resolved by SDS-PAGE and immuno-blotting using antibodies that specifically recognize PCAF;hGCN5, p300/CBP, TRRAP, and Cyclin T1-cdk9. Depending on the stability of Tat-associated TAR-RNA complexes, it may also be necessary to cross-link nucleoprotein interactions by treating extracts with formaldehyde in the presence of the biotinylated TAR-RNA riboprobe. Since trans-inhibition of WRN functions significantly inhibits HIV-1 LTR trans-activation and virus replication (see FIGS. 6 and 7), if WRN/Tat interactions modulate the recruitment of other transcriptional coactivators to the HIV-1 promoter, it is likely that these experiments may facilitate identification of essential cellular factors that contribute to HIV-1 replication and whose activities are dependent upon WRN. The biotinylated TAR-RNA probe may also be used in streptavidin-agarose pull-down assays to precipitate Tat/TAR-RNA complexes in transfected WRN^{-I-} fibroblasts, which lack functional WRN helicase, in order to directly assess the importance of WRN upon HIV-1 TAR-RNA complexes.

As interactions between HIV-1 Tat and the PCAF transcriptional coactivator enhance recruitment of PTEF-b (Cyclin T1-cdk9) complexes to TAR-RNA and stimulate RNA Pol II hyper-phosphorylation (Kiernan R E et al., EMBO J., 18:6106-6118; Bres V et al., EMBO J. 21:6811-6819; Zhou M et al., PNAS 100:12666-12671), it is likely that inhibition of WRN functions, through expression of WRN_{K577M}, may significantly influence Tat-PCAF interactions and RNA Pol II phosphorylation on the HIV-1 LTR. To determine whether inhibition of WRN results in changes of Tat-mediated RNA Pol II phosphorylation, ChIP analyses may be performed on PHA/hIL-2 stimulated HIV-1-infected H9_{HIV-1IIIB} lymphocytes, transfected with CMV-WRN_{K577M}, CMV-WRN, or an empty CβF vector control, using antibodies that bind phosphorylated-RNA Pol II (Santa Cruz Biotechnology). If WRN-recruitment by Tat to TAR-RNA facilitates interactions with other Tat-associated factors, such as PCAF and Cyclin T1-cdk9, or “recycling” of Tat through dissociation of Tat/TAR-RNA complexes, then inhibition of WRN should diminish RNA Pol II-phosphorylation on the HIV-1 LTR coincident with repression of transcriptional activity. Indeed, inhibition of WRN functions, through expression of the trans-dominant-negative WRN_{K577M} mutant protein, potently represses basal and Tat-activated HIV-1 LTR transcriptional activity, prevents intracellular synthesis of p24^{Gag} and inhibits viral replication in HIV-infected H9_{HIV-1IIIB} lymphocytes. These experiments will address whether WRN/Tat/TAR-RNA interactions contribute to RNA Pol II-phosphorylation which could enhance HIV-1 transcriptional elongation and trans-activation.

Further, inflammatory immune-signals, such as TGF-β-, TNF-α-, IL-2-receptor-signaling, transcriptionally activate enhancer elements (NF-κB, NF-AT, SP-1) within the HIV-1 promoter to stimulate viral replication in infected cells and latent tissue HIV-reservoirs (e.g., lymphatic tissues, spleen, CNS (Bohnlein E et al., Cell 53:827-836; Duh E J et al., PNAS 86:5974-5978; Osborn L et al., PNAS 86:2336-2340Li J M et al., Mol. Cell. Biol. 18:110-121; Poli G et al., PNAS 91:108-112). The WRN helicase has been demonstrated to serve as a substrate for phosphorylation by Abl tyrosine kinase, ATR/ATM kinases, and DNA-dependent protein kinase (DNA-PK)(Karmakar P et al., J. Biol. Chem. 277:18291-18302; Pichierri P et al., Oncogene 22:1491-1500; Cheng W H et al., Mol. Cell. Biol. 23:6385-6395; Yannone S M et al., J. Biol. Chem. 276:38242-38248). Phosphorylation of WRN regulates its subcellular nuclear distribution (nucleolar; nucleoplasmic (Pichierri P et al., Oncogene 22:1491-1500; Cheng W H et al., Mol. Cell. Biol. 23:6385-6395), and it is likely that cellular immune-signals modulate WRN functions through phosphorylation. WRN co-localizes with Tat in nucleoli or the nucleoplasm of HIV-1-infected Neuro-AIDS patient tissues by immunofluorescence-laser confocal microscopy (Example 6). ChIP analyses may be used to determine whether TGF-β-, TNF-α-, IL-2-signaling influences recruitment of the WRN helicase to Tat/TAR-RNA complexes on the HIV-1 LTR in treated H9_{HIV-1IIIB} lymphocytes. Effects of inflammatory immune-signals upon the recruitment of transcriptional accessory factors, including PCAF, Cyclin T1-cdk9, p300/CBP, TRRAP, to the HIV-1 LTR may also be investigated through these studies. Finally, the influence, if any, of TGF-β-, TNF-α-, IL-2-receptor-signals on WRN-mediated transcriptional activation from the HIV-1 LTR and viral replication in infected lymphocytes, as well as the ability of trans-dominant-negative WRN_{K577M} to repress HIV-1 LTR-driven transcription may be determined. These experiments may further reveal the nature of the molecular events involved in HIV-1 transcription and gene expression by characterizing novel LTR nucleoprotein interactions with the WRN helicase, which is essential for basal and activated HIV replication.

### Example 9

Initially, linker-scanning Ala-Ala-substitution mutations may be introduced into the HIV-1 Tat coding sequence to identify specific amino acid residues that interact with WRN. Further, WRN/Tat/TAR-RNA ternary complexes may be photochemically-crosslinked and subjected to tryptic endopeptidase digestion. Residues within WRN or Tat that contact TAR-RNA may be identified by radiolabeling the TAR-RNA and through Edman-degradation automated sequence analyses or mass-spectrometry of radioactive tryptic peptide fragments. Affinity chromatography, using various immobilized domains of HIV-1 Tat or WRN, may be used to directly assess protein-protein interactions between Tat/WRN. This Specific Aim will also determine the effects of immune/cytokine-signaling upon Tat/WRN interactions on TAR-RNA. Once the critical amino acid and nucleic acid contacts between HIV-1 Tat and WRN on TAR-RNA have been resolved, structural biochemical approaches (X-ray crystallography; Nuclear Magnetic Resonance (NMR) spectroscopy) may be used to model these interactions and may facilitate the design of small-molecule inhibitors to prevent formation of WRN/Tat/TAR-RNA complexes and inhibit HIV replication.

The essential protein-protein and protein-nucleic acid contacts between WRN/Tat or WRN/Tat/TAR-RNA may be identified through mutagenesis and structural biochemical analyses (nuclear magnetic resonance (NMR) spectroscopy; X-ray crystallography), to facilitate the design of small molecule inhibitors which may lead to the development of new effective anti-HIV/AIDS therapies.

WRN trans-activates the HIV-1 LTR, dependent upon WRN-associated helicase activity and TAR-RNA. A transcriptionally-inactive TatK28A/K50A mutant, defective for binding TAR-RNA (Kiernan R E et al., EMBO J. 18:6106-6118), inhibited WRN-mediated LTR trans-activation suggesting that WRN/Tat may directly interact. WRN has been detected in chromatin-remodeling complexes, containing Tat, Cyclin T1, PCAF, p300, and TRRAP, assembled on HIV-1 LTR sequences in the vicinity of TAR-RNA (Nuc 1). Indeed, these data indicate that WRN interacts with Tat/TAR-RNA nucleoprotein complexes recruited to the HIV-1 promoter. In addition, WRN is essential for HIV-1 transcriptional activity and virus replication. Identification of critical amino acid residues and nucleic acid contacts which participate in WRN/Tat/TAR-RNA interactions through mutagenesis and structural biochemical studies will significantly facilitate the design of small molecule inhibitors that may lead to the development of novel anti-HIV/AIDS therapies. Such experiments may also determine the influence of immune/cytokine-signaling upon WRN-Tat interactions on TAR-RNA.

Valuable information regarding protein-protein and nucleic acid interactions that contribute to WRN/Tat/TAR-RNA complex formation and HIV-1 LTR trans-activation may be gained through the experiments described in the present example. Deletion mutants, encompassing various WRN functional domains (amino acid residues 1-240, exonuclease domain; residues 568-859, helicase domain) or spanning the entire WRN coding sequence (residues 1-568, 568-1432, 859-1432), may be generated as glutathione-S-transferase (GST) fusion proteins (GST-WRN or GST-WRNΔ mutants) and expressed/purified in E. coli, BL21, bacteria. These proteins may be used in GST-pull-down assays or affinity chromatography experiments, using either nuclear extracts prepared from HIV-1-infected H9_{HIV-1IIIB} lymphocytes or purified recombinant Tat proteins, to broadly identify amino acid residues within WRN that interact with HIV-1 Tat. GST-pull-down assays may be performed by mixing and incubating purified recombinant GST-WRN or GST-WRNΔ mutant proteins with lymphocyte nuclear extracts expressing Tat or purified HIV-1 Tat protein. Glutathione-Sepharose-4B (Amersham-Pharmacia Biotech) may be added and bound GST-WRN/Tat complexes may be precipitated by centrifugation and analyzed by SDS-PAGE and immunoblotting using anti-HIV-1 Tat rabbit polyclonal antibody. The GST-WRN fusion proteins will also be immobilized on a glutathione-Sepharose-4B affinity column and nuclear extracts prepared from HIV-1-infected cells or purified recombinant HIV-1 Tat protein may be added to the column and bound GST-WRN/Tat complexes may be eluted using 10 mM reduced glutathione buffer and analyzed by immunoblotting as described. Once the Tat-interacting domain(s) within WRN has been defined, double alanine (Ala-Ala) substitution mutations may be introduced at various locations within the truncated WRN coding sequence to identify specific amino acids that are essential for interactions with Tat. Moreover, by testing the abilities of GST-WRN deletions or alanine-substitution mutant proteins to interact with a radioactive HIV-1 TAR-RNA element riboprobe, unique residues in WRN responsible for directly binding the TAR-RNA stem-loop may be identified.

To identify residues within HIV-1 Tat that recruit the WRN helicase to Tat/TAR-RNA promoter complexes, linker-scanning Ala-Ala-substitution mutations may be introduced into the GST-HIV-1 Tat coding sequence (Kiernan R E et al., EMBO J. 18:6106-6118) to identify specific amino acid residues that interact with WRN. Glutathione-S-transferase-pull-down assays may be performed using Jurkat nuclear extracts to delineate the domain(s) within Tat that interact with WRN. HIV-1 Tat-derived mutants, which are defective for WRN-binding, identified through these experiments will then be subcloned into an RSV-HIV-1 Tat construct and expressed in Jurkat lymphocytes to assess their capacities to trans-activate the HIV-1 LTR and interact in promoter complexes containing WRN, Cyclin T1, PCAF, p300, TRRAP, as determined by ChIP analyses (Example 5). These mutants will also be expressed in T-cell-lines containing HIV-1^{ΔTat} proviruses (Sadaie M R et al., New Biol. 2:479-486; Popik W et al., Virology 189:435-447) to determine their abilities to promote viral replication. HIV-1 replication and virus production may be quantified by measuring intracellular and extracellular p24^{Gag} levels by ELISAs and immuno-blotting with anti-HIV-1 p24^{Gag} monoclonal antibody (Example 5).

Alternatively, residues within HIV-1 Tat and WRN which are essential for WRN/Tat/TAR-RNA nucleoprotein complex formation may be identified by photochemically-crosslinking purified recombinant WRN and Tat proteins to a synthetic radiolabeled TAR-RNA probe. The ternary complexes may then be subjected to tryptic endopeptidase digestion and residues within WRN or Tat that contact TAR-RNA may be identified by Edman-degradation automated sequence analyses or mass-spectrometry of radioactive tryptic peptide fragment. Once the critical amino acid and nucleic acid contacts between HIV-1 Tat and WRN on TAR-RNA have been resolved, structural biochemical experiments (X-ray crystallography; NMR spectroscopy) may be performed using purified recombinant proteins or synthetic peptides of interacting domains to model these interactions and facilitate the design of small molecule inhibitors to prevent formation of WRN/Tat/TAR-RNA complexes and inhibit HIV replication.

As will be understood by those of ordinary skill in the art, other equivalent or alternative methods, and/or compositions for reducing HIV transcription and/or replication may be envisioned without departing from the essential characteristics thereof. For example, methods and dosages may be scaled to cells and/or subjects with differing in maturity (e.g., children and adults) and/or viral load and subjects with additional infections or conditions and/or symptoms. In addition, methods and dosages may be adapted to fluctuations over time (e.g., monthly or seasonal). These equivalents and alternatives along with obvious changes and modifications are intended to be included within the scope of the present disclosure. For example, values and/or range endpoints provided are not intended to be rigid limits for all embodiments. Moreover, one of ordinary skill in the art will appreciate that no single embodiment, use, and/or advantage is intended to universally control or exclude other embodiments, uses, and/or advantages. For example, a medical practitioner may deem circumstances to warrant giving preference to one over another. Accordingly, the foregoing disclosure is intended to be illustrative, but not limiting, of the scope of the disclosure as illustrated by the following claims.

What is claimed is:

1. A method of inhibiting human immunodeficiency virus type 1 (HIV-1) transcription or replication in a subject, comprising:
contacting a cell of the subject with a nucleic acid, polypeptide, or organic molecule that inhibits WRN-associated helicase activity in an amount and for a time sufficient to interfere with WRN-associated helicase activity in the cell.

2. The method of claim 1, wherein the nucleic acid encodes a trans-dominant-negative mutant.

3. The method of claim 2, wherein the trans-dominant negative mutant is WRN_{K577M}.

4. The method of claim 1 further comprising administering a pharmaceutically effective amount of an anti-retroviral agent selected from the group consisting of a viral reverse transcriptase inhibitor, a protease inhibitor, an integrase inhibitor, and a fusion inhibitor.

5. The method of claim 1, wherein the subject is a human.

6. A method of inhibiting human immunodeficiency virus type 1 (HIV-1) transcription or replication in a cell, comprising:
contacting the cell with a nucleic acid, polypeptide, or organic molecule that inhibits WRN-associated helicase activity in an amount and for a time sufficient to interfere with WRN-associated helicase activity in the cell.

7. The method of claim 6, wherein the nucleic acid encodes a trans-dominant-negative mutant.

8. The method of claim 7, wherein the trans-dominant negative mutant is WRN_{K577M}.

9. The method of claim 6 further comprising contacting the cell with a pharmaceutically effective amount of an anti-retroviral agent selected from the group consisting of a viral reverse transcriptase inhibitor, a protease inhibitor, an integrase inhibitor, and a fusion inhibitor.

10. A method of identifying an anti-HIV drug lead comprising:
contacting a test molecule with a cell including:
a first nucleic acid having an HIV-1 long terminal repeat promoter operably linked to a reporter gene; and
a second nucleic acid having a constitutive promoter operably linked to the Werner's Syndrome helicase gene; and
cultivating the cell under conditions that permit expression of the reporter gene and the helicase gene in the absence of a candidate drug molecule,
wherein the reporter gene product is lethal to the cell and the presence of an anti-HIV candidate drug is indicated by survival of the cell.

11. The method of claim 10, wherein the cell further comprises a third nucleic acid having a constitutive promoter operably linked to an HIV-1 Tat gene, wherein the constitutive promoter of the third nucleic acid may be the same or different from the constitutive promoter of the second nucleic acid.

12. The method of claim 10, wherein the method is performed as a high throughput screen.

13. A method of identifying a molecule that interferes with WRN helicase-HIV-1 Tat interaction, comprising:
contacting a test molecule with a cell including:
a first nucleic acid having an HIV-1 long terminal repeat promoter operably linked to a reporter gene;
a second nucleic acid having a constitutive promoter operably linked to the Werner's Syndrome helicase gene; and
a third nucleic acid comprising a constitutive promoter operably linked to an HIV-1 Tat gene, wherein the constitutive promoter of the third nucleic acid may be the same or different from the constitutive promoter of the second nucleic acid;
cultivating the cell under conditions that permit expression of the reporter gene and the helicase gene in the absence of the test molecule,
detecting the reporter gene expression in the presence of the test molecule; and
comparing the reporter gene expression in the presence of the test molecule with reporter gene expression in the absence of the test molecule,
wherein reduced reporter gene expression in the presence of the test molecule relative to reporter gene expression in the absence of the test molecule indicates inhibition of WRN helicase-HIV-1 Tat interaction.

14. The method of claim 13, wherein the method is performed as a high throughput screen.

15. The method of claim 13, wherein the reporter gene encodes a protein selected from the group consisting of β-galactosidase, β-glucuronidase, an autofluorescent protein, glutathione-S-transferase, luciferase, horseradish peroxidase, chloramphenicol acetyltransferase, derivatives or fragments thereof, and any combination thereof.

16. A method of identifying a molecule that affects WRN helicase-HIV-1 Tat interaction upon phosphorylation of RNA Polymerase II, comprising:
contacting a test molecule with a cell including:
a first nucleic acid having an HIV-1 long terminal repeat promoter operably linked to a reporter gene;
a second nucleic acid having a constitutive promoter operably linked to the Werner's Syndrome helicase gene; and
a third nucleic acid having a constitutive promoter operably linked to an HIV-1 Tat gene, wherein the constitutive promoter of the third nucleic acid may be the same or different from the constitutive promoter of the second nucleic acid;
cultivating the cell under conditions that permit expression of the reporter gene and the helicase gene in the absence of the test molecule;
cross-linking nucleoprotein transcription complexes bound to the HIV-1 promoter;
extracting chromatinized total genomic DNA from the cell;
fragmenting the chromatinized total genomic DNA into oligonucleosomal DNA fragments; and
contacting the oligonucleosomal DNA fragments with a phospho-RNA Polymerase II-specific antibody; and
comparing the phosphorylation of RNA Polymerase II in the presence of the test molecule with phosphorylation of RNA Polymerase II in the absence of the test molecule,
wherein a difference in phosphorylation of RNA Polymerase II in the presence of the test molecule relative to phosphorylation of RNA Polymerase II in the absence of the test molecule indicates that the test molecule affects WRN helicase-HIV-1 Tat interaction upon phosphorylation of RNA Polymerase II.
